(19) United States
(12) Patent Application Publication
(10) Pub. No.: US 2025/0314635 A1
(43) Pub. Date: Oct. 9, 2025

(54) PREPARATION SYSTEMS FOR WELL BOTTOM COMPOSITE SCALE SAMPLES

(71) Applicant: CHONGQING UNIVERSITY OF SCIENCE AND TECHNOLOGY, Chongqing (CN)

(72) Inventors: Qianhua XIAO, Chongqing (CN); Shijie ZHU, Chongqing (CN); Zhuangzhuang HUANG, Chongqing (CN); Zhijun LI, Chongqing (CN); Zuping XIANG, Chongqing (CN); Lei LIU, Chongqing (CN); Zhengqi SU, Chongqing (CN); Peixian SHAO, Chongqing (CN); Na DONG, Chongqing (CN); Jiahao LI, Chongqing (CN); Ling SHI, Chongqing (CN)

(73) Assignee: CHONGQING UNIVERSITY OF SCIENCE AND TECHNOLOGY, Chongqing (CN)

(21) Appl. No.: 19/015,732

(22) Filed: Jan. 10, 2025

(30) Foreign Application Priority Data
Apr. 3, 2024 (CN) 202410398080.4

(51) Int. Cl. G01N 33/28; E21B 47/00

(52) U.S. Cl. CPC G01N 33/2823; E21B 47/006

(57) ABSTRACT

Preparation system for well bottom composite scale sample, comprising an analysis device, a processor, a formulation device, and an aging device. The processor is configured to: obtain actual scale sample of polymer injection well and reservoir parameter of reservoir in which the polymer injection well is located; perform qualitative analysis on the actual scale sample to determine flocculation type and scale sample parameter of the actual scale sample; perform quantitative analysis on the actual scale sample to determine components of the actual scale sample and content of each component; generate formulation instruction based on the flocculation type, the components, and the content of each component, and send the formulation instruction to the formulation device to formulate an artificial scale sample; and generate, based on the reservoir parameter, aging instruction, and send the aging instruction to the aging device to age the artificial scale sample to obtain composite scale sample.

### CROSS-REFERENCE TO RELATED APPLICATIONS

This application claims priority of Chinese Patent Application No. 202410398080.4, filed on Apr. 3, 2024, the entire contents of which are incorporated herein by reference.

### TECHNICAL FIELD

The present disclosure generally relates to the field of oil and gas extraction technology, and in particular, to a preparation system for a well bottom composite scale sample.

### BACKGROUND

Polymer flooding technology is a tertiary oil recovery technology that realizes the increase of crude oil production by injecting a polymer solution into the formation for oil recovery.

In the current application of polymer flooding, the injection capacity of the polymer injection well is significantly reduced due to the clogging of the polymer injection well caused by flocculent polymers. The flocculated polymer solution has high viscosity and poor flowability, and is very easy to be retained in the near-well zone, which constantly wraps all kinds of solid particles of impurities (e.g., hydration-expanded clay particles, dislodged sand and gravel particles, and all kinds of pipeline solids of the injection well) to form plugs with larger hydrodynamic sizes, thereby drastically reducing the seepage capacity of the injected fluid, leading to the transformation of the injection well into an inefficient injection well, thus affecting the oilfield's injection and extraction balance, leading to a decline in the fluid production capacity of the production well.

Under the environmental conditions of polymer injection well application, the scaling mechanism of flocculent polymer reacting with other solid particles to form a composite scale is still unknown, so it is of great importance for the research and development of targeted polymer systems to further reveal the polymer flocculent plugging scaling mechanism. However, sampling of the plugs of the polymer injection well is only done during single-well workover operations, each single-well workover has high costs and a long construction period, and an amount of the scale sample taken out each time is small, so it is difficult to satisfy the needs of experimental research.

Therefore, it is desired to provide a preparation system for a well bottom composite scale sample to prepare and obtain a large number of composite scale samples of a well organic matter system that meets the needs of experimental research.

### SUMMARY

The purpose of the present disclosure is to provide a preparation system for a well bottom composite scale sample and a composite scale sample of an organic matter system, to prepare a composite scale sample of a polymer flocculent indoors, to solve the problems of difficult sampling in polymer injection well and insufficient experimental samples of scale samples, and to provide support for accurately developing the cause of the scale sample and the scale mechanism.

One or more embodiments of the present disclosure provide a preparation system for a well bottom composite scale sample. The preparation system may include an analysis device, a processor, a formulation device, and an aging device. The processor may be configured to perform operations including obtaining an actual scale sample of a polymer injection well and a reservoir parameter of the reservoir in which the polymer injection well is located, performing a qualitative analysis on the actual scale sample using the analysis device to determine a flocculation type and a scale sample parameter of the actual scale sample; performing a quantitative analysis on the actual scale sample by the analysis device to determine the components of the actual scale sample and the content of each component of the actual scale sample, generating a formulation instruction based on the flocculation type, components, and content of the actual scale sample and sending the formulation instruction to the formulation device to cause the formulation device to formulate an artificial scale sample, and generating an aging instruction based on the reservoir parameter and sending the aging instruction to the aging device to cause the aging device to age the artificial scale sample to obtain a composite scale sample.

One or more embodiments of the present disclosure provides a composite scale sample of organic matter system, which is relatively close to the actual scale sample in terms of composition and flocculation type and can be regarded as an artificial replica of the actual scale sample. It solves the problem of insufficient material for injection well plugging mechanism research and indoor validation of deplugging technology caused by the small number and high cost of actual scale sample and differences of different scale samples in the existing technology, which is conducive to the promotion of the study of the plugging mechanism of injection well and the indoor verification experiments of the deplugging technology.

Some embodiments of the present disclosure include at least the following beneficial effects: (1) analyzing and judging the type of formed scale samples and their compositions based on the actual obtained scale samples, and formulating and preparing the composite scale samples based on polymer flocculent indoors according to the actual reservoir parameters, solving the problems of difficult sampling of polymer injection well and insufficient experimental samples of the scale samples, and providing support for accurately developing the cause of the scale samples and the descaling mechanism. (2) composite scale samples mainly composed of organic matter system, which is prepared by the system, and the actual obtained composite scale samples has good consistency, which may meet the needs of experimental research and provide technical support for analyzing the injection of plugging mechanism and deplugging technology research. (3) Since the composite scale sample is obtained through replication indoors, they are low-cost and time-efficient, which is conducive to the reduction of the cost of research on the cause of scale samples, the plugging mechanism of injection well, the mechanism of descaling, and the design and validation of descaling programs.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, wherein:

FIG. 1 is a diagram illustrating exemplary modules of a preparation system for a well bottom composite scale sample according to some embodiments of the present disclosure;

FIG. 2 is a flowchart illustrating an exemplary preparation system for a well bottom composite scale sample according to some embodiments of the present disclosure;

FIGS. 3 and 4 are XRD patterns of the actual scale samples (samples) according to some embodiments of the present disclosure;

FIGS. 5-14 are SEM images from different orientations and energy dispersive X-ray Spectroscopy (EDS) maps at different points of the actual scale samples (samples) according to some embodiments of the present disclosure;

FIG. 15 is an infrared spectrum of the organic matter separated from the actual scale sample (sample) according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. It should be understood that the purposes of these illustrated embodiments are only provided to those skilled in the art to practice the application, and not intended to limit the scope of the present disclosure. Unless obviously obtained from the context or the context illustrates otherwise, the same numeral in the drawings refers to the same structure or operation. It will be understood that the terms “system,” “device,” and/or “equipment” used herein are one method to distinguish different components, elements, parts, sections, or assemblies of different levels in ascending order. However, the terms may be displaced by other expressions if they may achieve the same purpose.

The terminology used herein is for the purposes of describing particular examples and embodiments only and is not intended to be limiting. As used herein, the singular forms “a,” “an,” and “the” may be intended to include the plural forms as well, unless the context clearly indicates otherwise. It will be further understood that the terms “include” and/or “comprise,” when used in this disclosure, specify the presence of integers, devices, behaviors, stated features, steps, elements, operations, and/or components, but do not exclude the presence or addition of one or more other integers, devices, behaviors, features, steps, elements, operations, components, and/or groups thereof.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

The technical solutions of the present disclosure will be clearly and completely described below in connection with embodiments, and it is clear that the embodiments described are a part of the embodiments of the present disclosure and not all of the embodiments. Based on the embodiments in the present disclosure, all other embodiments obtained by a person of ordinary skill in the art without making creative labor fall within the scope of protection of the present disclosure.

Scale sample is the basis for studying plugging mechanism and deplugging scheme of the injection well. However, the scale samples in the prior art not only depend on the sampling quality of the scale samples, but also have a long collection time and are expensive, which makes it difficult to advance the study of plugging mechanism and deplugging scheme of the injection well because of the insufficiency of the scale samples and the varying levels of scale sample quality, and to validate the plugging mechanism and the deplugging scheme.

In order to solve the problems of difficult sampling of polymer injection well and insufficient samples for scale sample experiments, some embodiments of the present disclosure provide a preparation system for a well bottom composite scale sample.

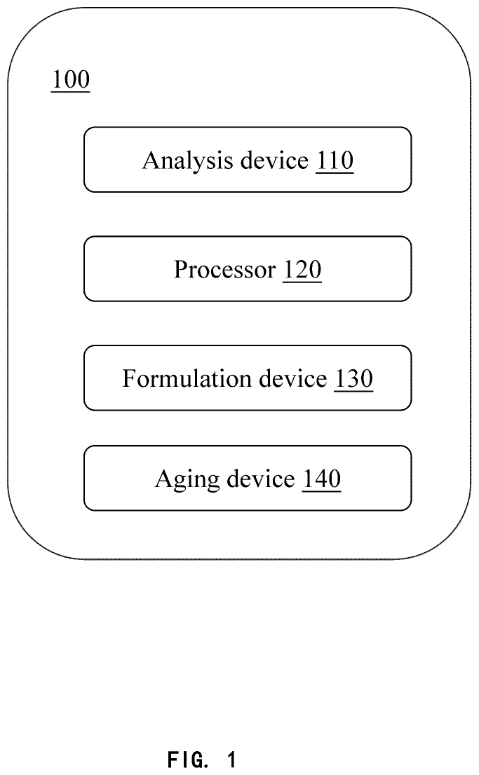

FIG. 1 is a diagram illustrating exemplary modules of a preparation system for a well bottom composite scale sample according to some embodiments of the present disclosure. As shown in FIG. 1, the preparation system for the well bottom composite scale sample 100 may include an analysis device 110, a processor 120, a formulation device 130, and an aging device 140.

The analysis device 110 refers to a device that analyzes a sample such as a scale sample. In some embodiments, the analysis device includes, but is not limited to, an X-ray diffractometer, a chromatograph, a scanning electron microscope, a densitometer, or the like. The X-ray diffractometer is configured to obtain an XRD pattern of the sample; the chromatograph is configured to obtain information such as the composition and content of the sample; the scanning electron microscope is configured to analyze the sample in terms of an energy spectrum; and the densitometer is configured to obtain the density of the sample.

In some embodiments, the analysis device 110 determines, by qualitative analysis, the flocculation type of the actual scale sample and the parameters of the scale sample; and determines, by quantitative analysis, the components of the actual scale sample and the content of each component.

The processor 120 refers to a core component of a computer system that is responsible for receiving, processing, and calculating input data, and generating outputs based on predefined programs and logic.

In some embodiments, the processor 120 is configured as a key component for executing program instructions and processing data. The processor may process and perform one or more of the functions described in this disclosure. For example, the processor may obtain an actual scale sample of the polymer injection well and a reservoir parameter of the reservoir in which the polymer injection well is located; generate, based on the flocculation type of the actual scale sample, the components of the actual scale sample, and the content of each of the components, a formulation instruction and send the formulation instruction to the formulation device.

In some embodiments, the processor may also generate aging instruction based on the reservoir parameter and send the aging instruction to the aging device.

The formulation device 130 refers to an apparatus for mixing and formulating an artificial scale sample according to requirements. In some embodiments, the formulation device includes, but is not limited to, a feeding device, a metering device, a stirrer, a mixing tank, or the like.

In some embodiments, the formulation device is configured to formulate an artificial scale sample based on the formulation instruction and transmit the artificial scale sample to the aging device.

The aging device 140 is an apparatus for performing aging treatment on the artificial scale sample. In some embodiments, the aging device includes a loading device, a thermostat, or the like.

In some embodiments, the aging device is configured to age the artificial scale sample based on aging instruction to obtain a composite scale sample.

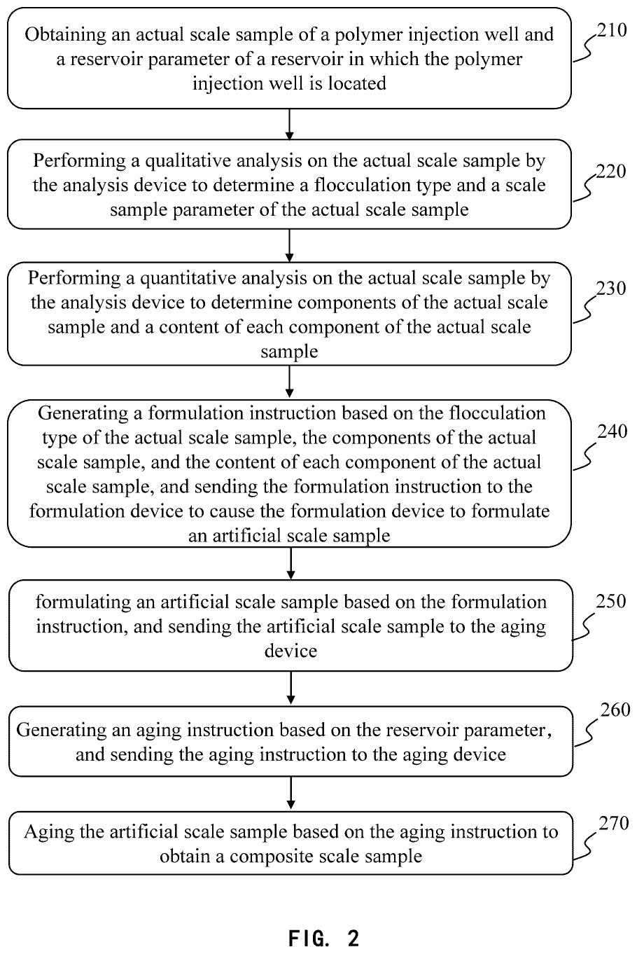

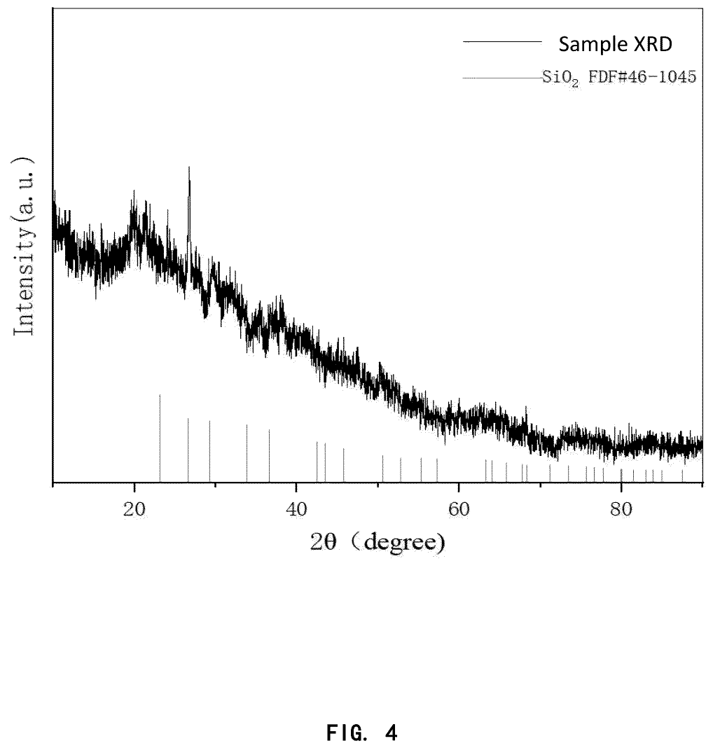

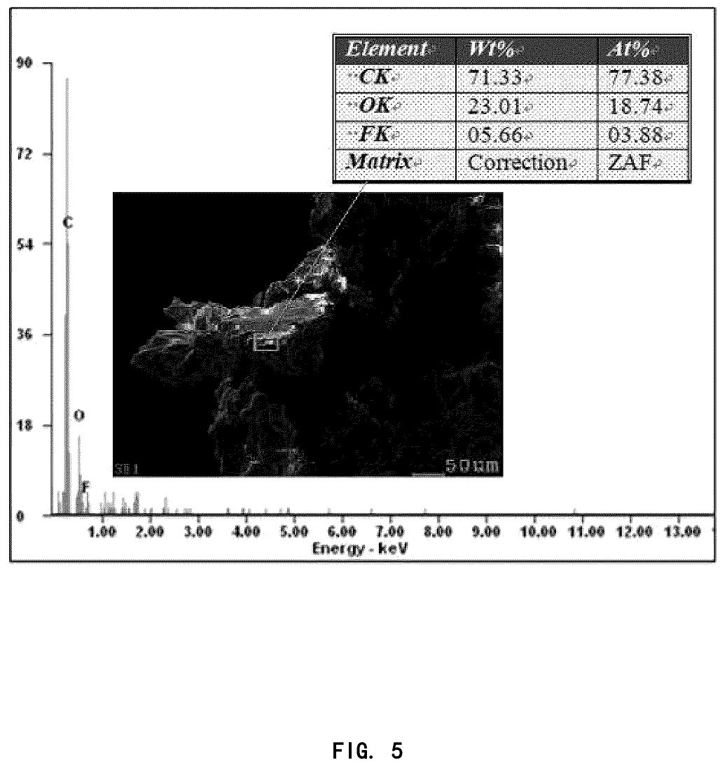

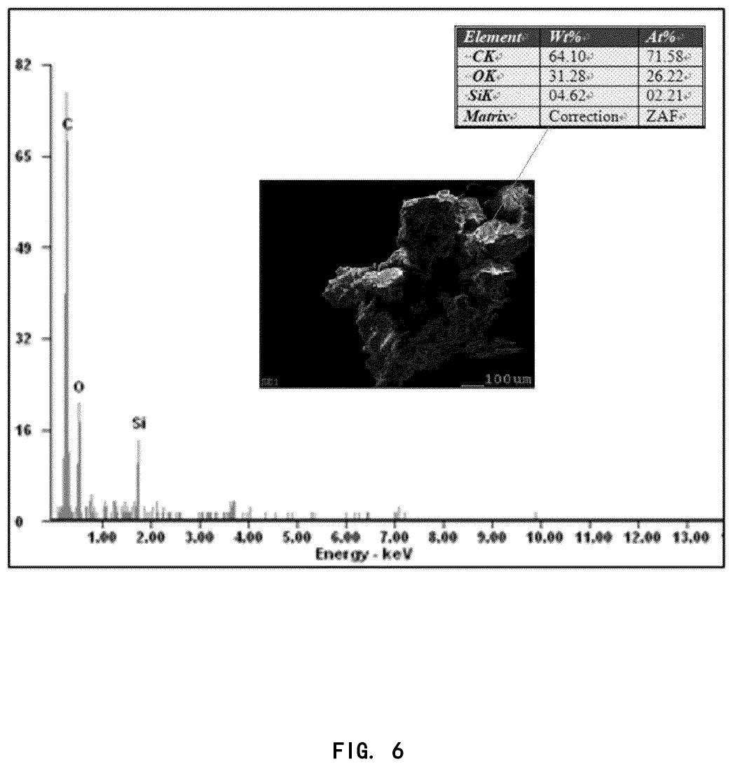

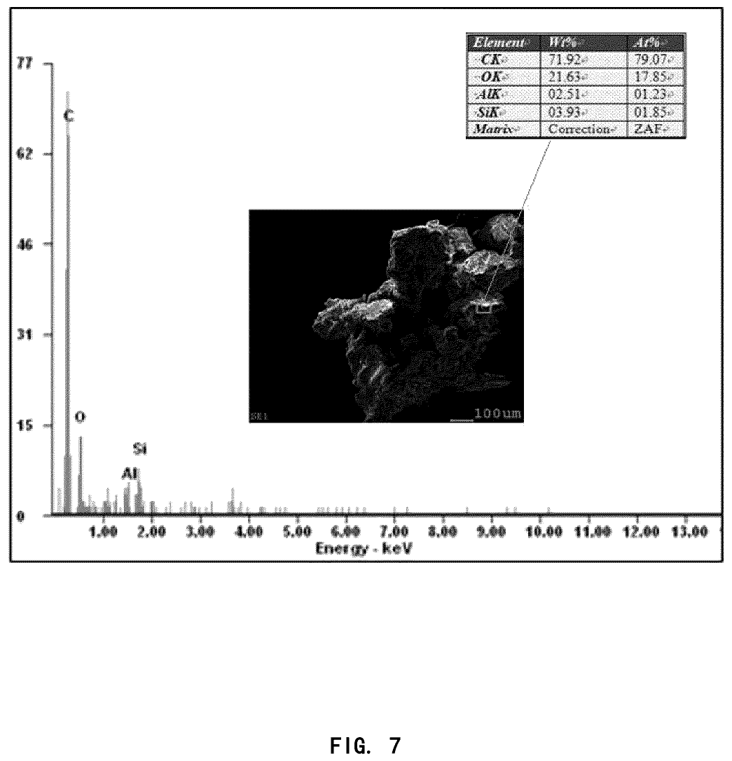

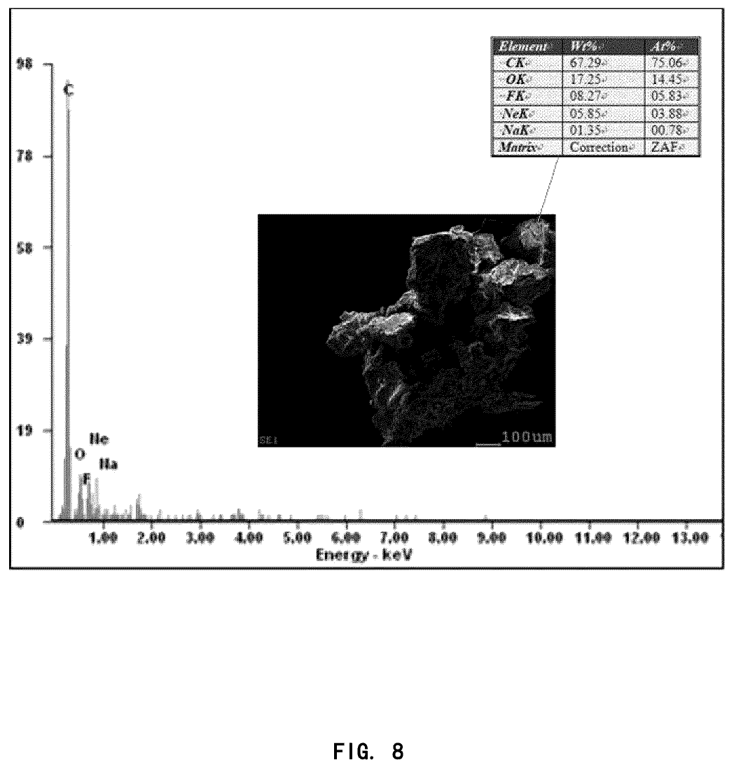

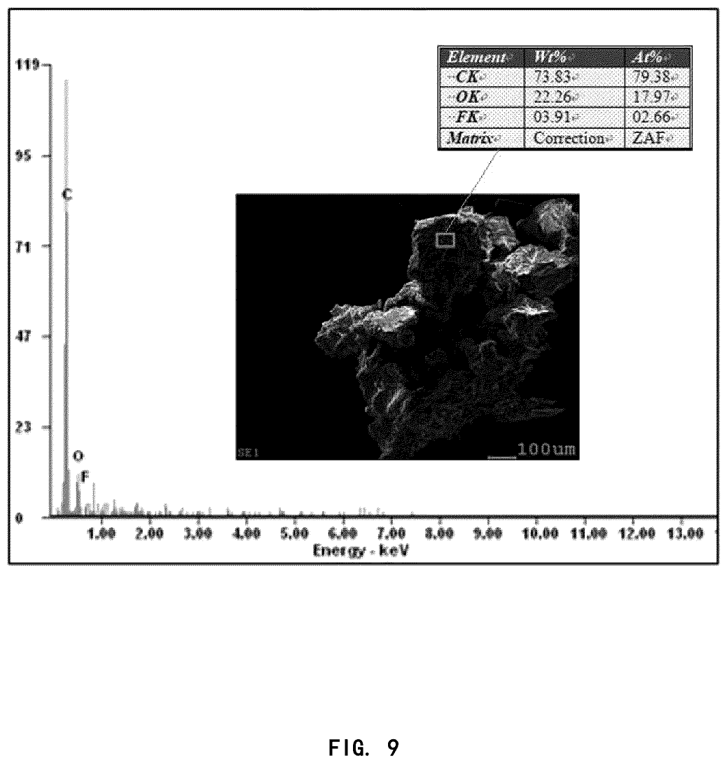

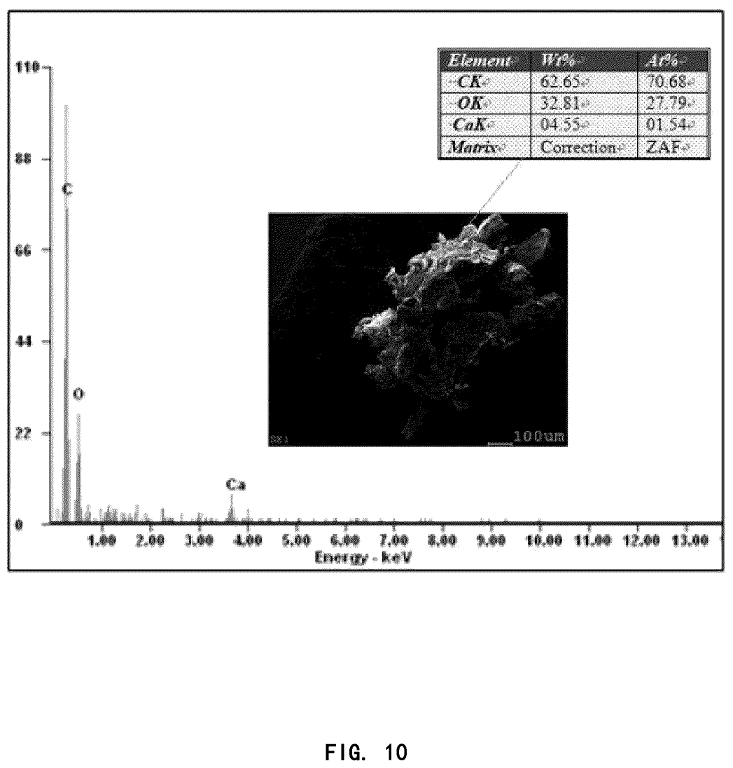

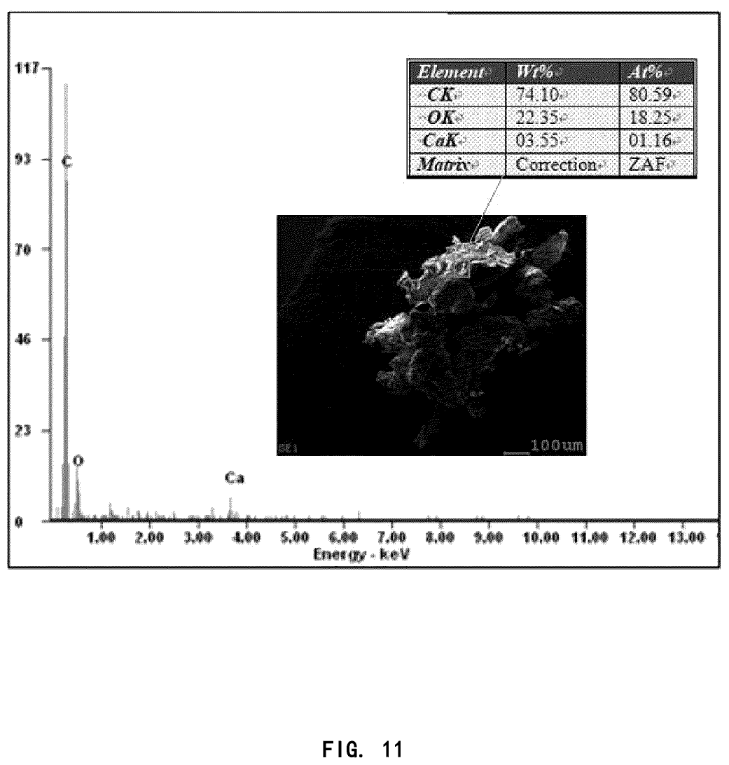

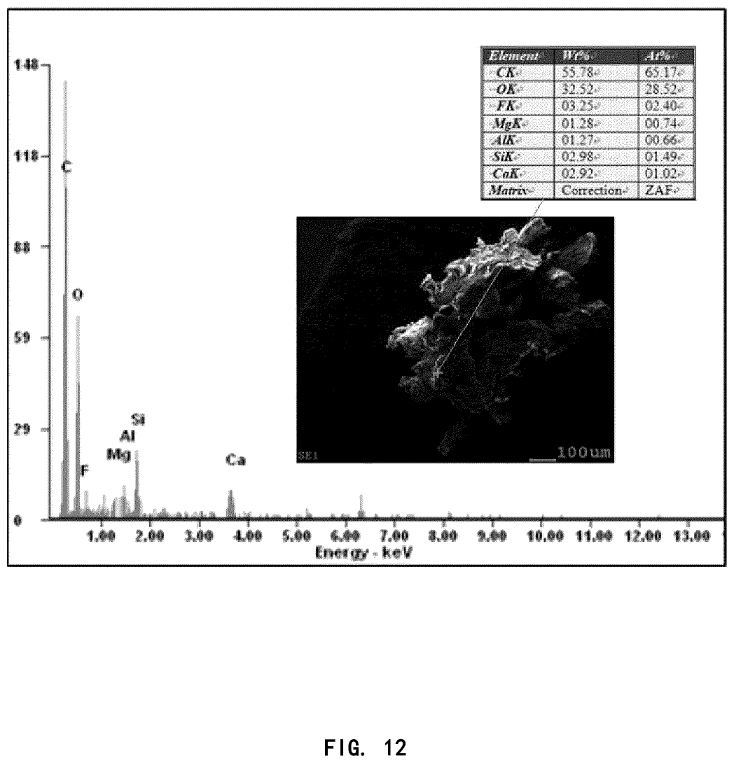

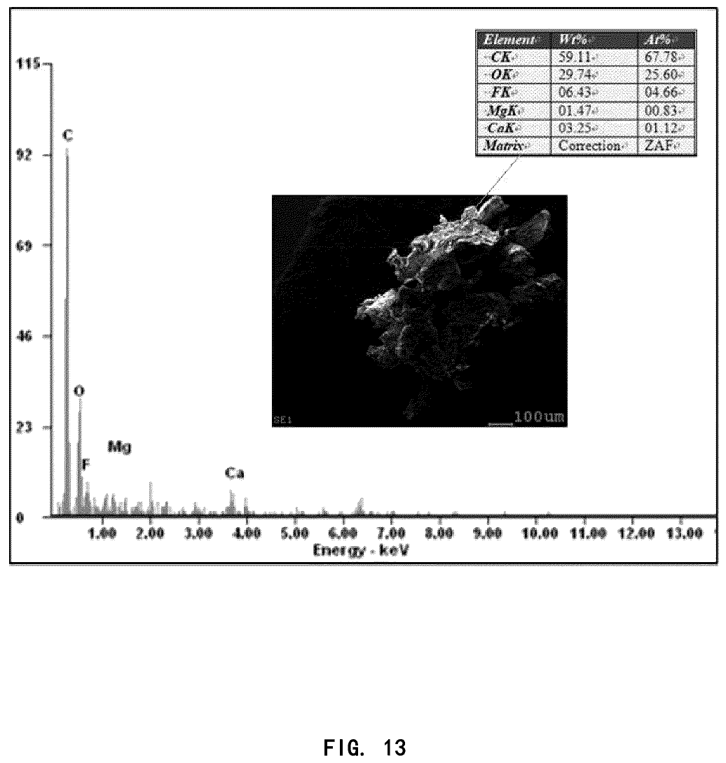

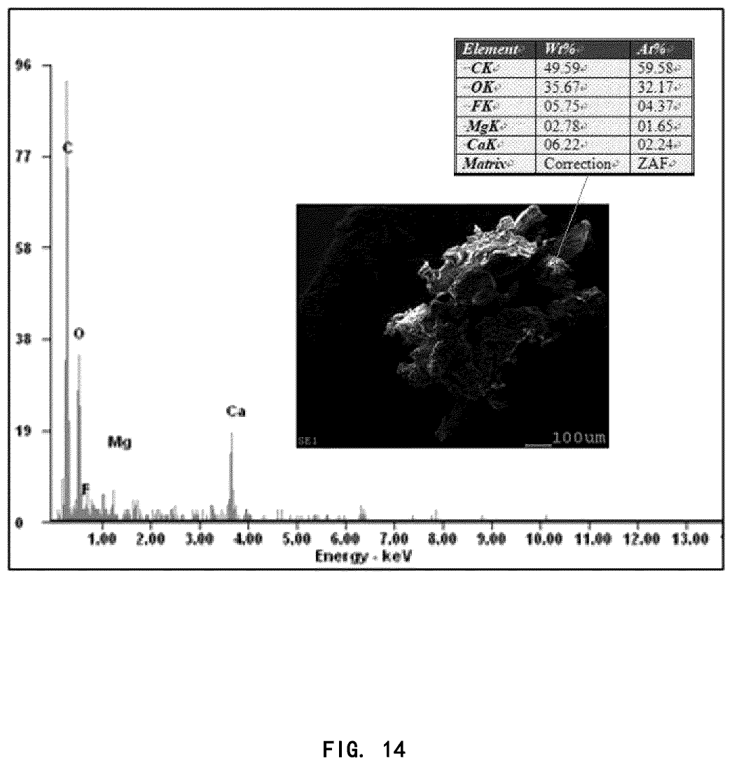

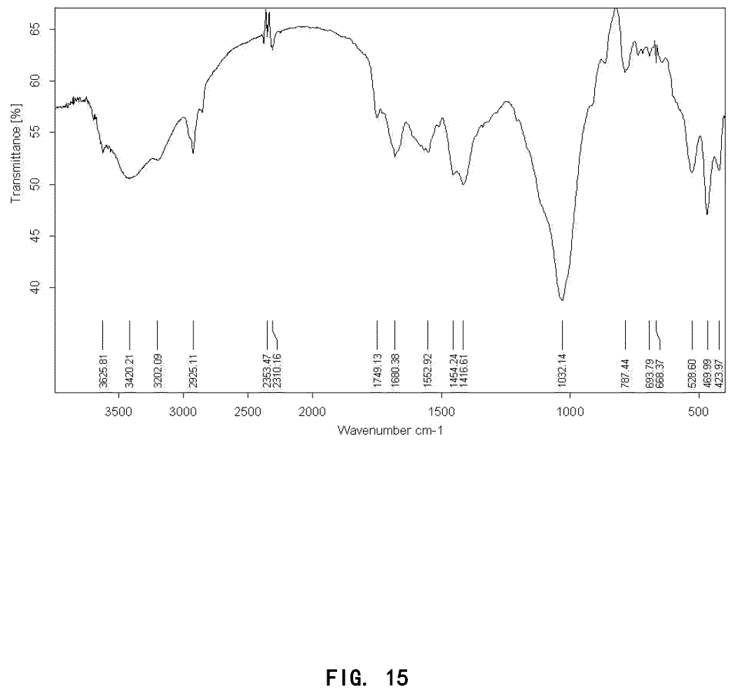

More about the relevant functions of each of the above devices or apparatus may be found in FIGS. 2-15 and their related descriptions.

In some embodiments of the present disclosure, based on the coordinated operation of various apparatus or devices in the preparation system for the well bottom composite scale sample 100, it is possible to realize the replication of the actual scale sample several times indoors, which provides sufficient composite scale samples for the study of scale sample causes, injection well plugging mechanisms, descaling mechanisms, and other operations.

It should be noted that the above description of the preparation system for the well bottom composite scale sample and modules is provided only for descriptive convenience and does not limit the present disclosure to the scope of the cited embodiments. It is to be understood that for a person skilled in the art, with an understanding of the principle of the system, it may be possible to arbitrarily combine the modules or form a subsystem to be connected to the other modules without departing from this principle.

FIG. 2 is a flowchart illustrating an exemplary preparation system for a well bottom composite scale sample according to some embodiments of the present disclosure. As shown in FIG. 2, process 200 includes the following operations.

Operation 210, obtaining an actual scale sample of a polymer injection well and a reservoir parameter of a reservoir in which the polymer injection well is located.

The polymer injection well is an oil well used for injecting polymer. The reservoir parameter of the reservoir where the polymer injection well is located may reflect the structural properties of the reservoir.

The reservoir parameter is a parameter related to the geologic layer where the actual scale sample is located, which may reflect the actual situation within the reservoir. The reservoir parameter may include, but is not limited to, the porosity of the reservoir rock, permeability, and mineralization and ionic composition of the reservoir water.

In some embodiments, the reservoir parameter further includes the actual reservoir temperature and the actual reservoir pressure.

In some embodiments, the actual scale sample of the polymer injection well may be obtained through exploration, and the reservoir parameter may be obtained through devices such as temperature sensors, pressure sensors, or the like located within the reservoir. The processor may also obtain the reservoir parameter based on data such as the exploration report of the geological formation.

Operation 220, performing a qualitative analysis on the actual scale sample by the analysis device to determine a flocculation type and a scale sample parameter of the actual scale sample.

The qualitative analysis is an analytical manner configured to determine the composition of a scale sample.

The scale sample parameter is indicators that characterize the physicochemical features of the scale sample at the well bottom. In some embodiments, the scale sample parameter may include infrared spectral information (e.g., functional group types, etc.), X-ray diffraction information (e.g., crystal structure, grain size, etc.), density, grain size distribution, and other information of the scale sample. The processor may obtain an infrared spectrum of the sample, an XRD pattern, the composition and content of the sample, an energy spectrum, a density, etc., by using an analysis device such as an infrared spectrometer, X-ray diffractometer, chromatograph, scanning electron microscope, densitometer, etc.

In some embodiments, the scale sample parameter further includes the viscosity of the actual scale sample. The processor may obtain the scale sample parameter by an analysis device such as a viscometer.

The flocculation type may reflect the flocculation features of the scale sample. In some embodiments, the flocculation type may include a microbial flocculation system, a charge-neutralizing flocculation system, a gel flocculation system, or the like.

In some embodiments, the flocculation type may also include a partially oxidized degraded flocculation system, a consolidated conformance control system, and a poorly dissolved flocculation system.

The partially oxidized degraded flocculation system is a system in which the molecular chains of the polymer undergo incomplete degradation under oxidizing conditions, resulting in the formation of flocculent that contain a high number of metal cations and/or other strongly oxidizing substances.

The consolidated conformance control system is a system in which flocculating clusters are formed due to interactions between polymer and mineral particle and fouling, resulting in the formation of flocculent with a dense structure and a high gelling strength.

The poorly dissolved flocculation system refers to a flocculating system in which the polymer is not completely dissolved due to insufficient dissolving conditions of the polymer (e.g., inappropriate temperature, insufficient time, insufficient stirring, etc.), resulting in the formation of flocculated material with large aggregates on the inside and gelled flocculent on the outside.

In some embodiments of the present disclosure, by introducing a plurality of flocculation types, it is possible to accurately classify different types of scale sample, which may contribute to the accuracy of formulating the artificial scale sample in a subsequent operation; and the viscosity of the collected scale sample is included in the scale sample parameter, which may more accurately represent the impact of the viscosity.

The flocculation type may be determined in a plurality of ways. The processor may determine the flocculation type of the scale sample by microscopic observation and compositional analysis of the scale sample with analysis device such as chromatograph and scanning electron microscope (SEM). For example, when the scanning electron microscope finds that microorganisms are aggregated in the scale sample and include a large count of proteins, polysaccharides, and other secretions, the flocculation type may be determined to be a microbial flocculation system; and when the chromatographer finds that the scale sample is electrically neutral, the flocculation type may be determined to be a charge-neutralizing flocculation system.

In some embodiments, the processor may also determine the flocculation type of the actual scale sample based on the compositional qualitative analysis and the morphological qualitative analysis.

The compositional qualitative analysis is configured to analyze the types of various components in a scale sample. In some embodiments, the compositional qualitative analysis includes determining the flocculation type of the actual scale sample based on the components of the actual scale sample, the content of each component, and the content of specific elements or ions.

For example, when the compositional qualitative analysis indicates that the scale sample has a high content of strongly oxidizing substances, such as metal cations (trivalent iron ions, etc.) and/or other strongly oxidizing substances, the flocculation type is determined to be a partially oxidized degraded flocculation system.

The morphological qualitative analysis is configured to analyze the macroscopic and/or microscopic morphology of the flocculent in the scale sample. In some embodiments, the morphological qualitative analysis includes determining the flocculation type of the actual scale sample based on the macroscopic and/or microscopic morphology of flocculent in the actual scale sample. For example, when the morphological qualitative analysis shows that the inside and outside of the scale sample have different structures, such as a large mass of aggregates at the core and a gel on the outside, the flocculation type may be determined to be a poorly dissolved flocculation system; and when the morphological qualitative analysis shows that the inside and outside of the scale sample have a more homogeneous structure, and the compositional qualitative analysis shows that the content of strong oxidizing substances is low, the flocculation type is determined to be a consolidated conformance control system.

In some embodiments of the present disclosure, determining the flocculation type of the scale sample through compositional qualitative analysis and morphological qualitative analysis can comprehensively analyze the features of the scale sample based on the two dimensions of the chemical composition and the physical morphology, thereby precisely determining the flocculation type of the scale sample.

In some embodiments, the analysis device is further configured to determine the polymer composition of the flocculent in the actual scale sample through compositional qualitative analysis.

The polymer composition refers to the composition of the components of the flocculent in the scale sample. In some embodiments, the polymer composition includes the types and content of polymer monomers or oligomers, cross-linkers, initiators, and other additives.

The processor may determine the polymer composition of the flocculent through compositional qualitative analysis based on the analysis device. For example, the polymer composition may be determined by analyzing the flocculent through infrared spectroscopy, mass spectrometry, or the like, based on the analysis device.

Operation 230, performing a quantitative analysis on the actual scale sample by the analysis device to determine components of the actual scale sample and a content of each component of the actual scale sample.

The components of the actual scale sample include, but are not limited to, water content, oil content, organic content, mineral content, and metal ion content.

In some embodiments, the processor may perform quantitative analysis to determine the components of the actual scale sample and the content of each component by means of equipment such as a thermogravimetric analyzer (TGA), a dry distillation apparatus, a Soxhlet extractor, a chromatograph, a scanning electron microscope, or the like. For example, the Soxhlet extractor is used to extract the crude oil of the actual scale sample using n-hexane to determine the oil content of the actual scale sample, and the thermogravimetric analyzer is used to perform high-temperature thermal degradation at 650° C. to determine the content of organic matter of the actual scale sample; the dry distillation apparatus is used for dry distillation at 110° C. to determine the water content of the actual scale sample; the chromatograph is used for chromatographic analysis to determine the components of the actual scale sample and the content of each component, etc.

Operation 240, generating a formulation instruction based on the flocculation type of the actual scale sample, the components of the actual scale sample, and the content of each component of the actual scale sample, and sending the formulation instruction to the formulation device to cause the formulation device to formulate an artificial scale sample.

The formulation instruction is an instruction configured to formulating artificial scale sample.

In some embodiments, the formulation instruction may include the flocculation type of the actual scale sample, the polymer composition of the flocculent in the actual scale sample, the components of the actual scale sample, and the content of each component. In some embodiments, the processor may determine the formulation instruction by querying a first preset table based on the flocculation type of the actual scale sample, the components of the actual scale sample, and the content of each component. The first preset table includes a correspondence between the flocculation type of the actual scale sample, the components of the actual scale sample, and the content of each component and the formulation instruction. The first preset table may be determined based on historical data or priori experience.

In some embodiments, the processor is further configured to generate the formulation instruction based on the flocculation type of the actual scale sample, the polymer composition of the flocculent in the actual scale sample, the component of the actual scale sample, and the content of each component and send the formulation instruction to the formulation device. For example, the processor may determine, based on the components of the actual scale sample and the content of each component, the types of ingredients and the count of ingredients to be blended by the formulation device, determine the order of addition of the ingredients of the formulation device and the stirring time, etc. based on the flocculation type of the actual scale sample, and the polymer component of the flocculent in the actual scale sample, and then generate the formulation instruction and send the formulation instruction to the formulation device.

In some embodiments of the present disclosure, determining the polymer composition of the flocculent by compositional qualitative analysis and including the polymer composition in the formulation instruction allows for a comprehensive consideration of the effect of the composition of the polymer on the artificial scale sample, which helps to improve the quality of the prepared artificial scale sample and the matching degree of the prepared artificial scale sample with the actual scale sample.

Operation 250, formulating an artificial scale sample based on the formulation instruction and transmitting the artificial scale sample to the aging device.

In some embodiments, the formulation device may formulate the artificial scale sample in a plurality of ways. For example, the formulation device mixes all of the ingredients based on the formulation instruction and directly obtains an artificial scale sample containing flocculent through a one-pot synthesis manner.

In some embodiments, the formulation device may prepare a flocculent based on the flocculation type of the actual scale sample, the components of the flocculent in the actual scale sample, and the content of each component, and mix other ingredients into the prepared flocculent to obtain an artificial scale sample. Merely by way of example, ingredients such as water, crude oil, silica, metal ion salts, or the like may be added to the prepared flocculent in accordance with the formulation instruction, and the artificial scale sample is obtained by mixing.

In some embodiments, the formulation device may prepare a flocculent in the composite scale sample based on the flocculation type of the actual scale sample and the polymer composition of the flocculent in the actual scale sample; adjust the scale sample parameter of the flocculent based on the adjustment parameter; and add ingredients other than the flocculent into the flocculent based on the components of the actual scale sample and the content of each component, to obtain the artificial scale sample. For more details on the adjustment parameter, please refer to the relevant sections below.

In some embodiments, the water added in the preparation of the artificial scale sample is formation water actually collected within the reservoir. As the formation water usually also contains certain microscopic organisms such as bacteria, it is beneficial for the artificial scale sample to synchronize the microbial growth and reproduction process in the process of forming the actual scale sample in the formation during the aging process.

In some embodiments, the crude oil added in the preparation of the artificial scale sample is crude oil actually collected from the injection well.

In some embodiments, in order to make the artificial scale sample conform to the actual scale sample as much as possible, when preparing the artificial scale sample, the added solid materials such as silica and other solid materials have the same features in terms of particle size distribution, shape, and other features as those of the solid materials within the actual scale sample to maximize the replication of the actual scale sample.

Operation 260, generating an aging instruction based on the reservoir parameter, and sending the aging instruction to the aging device.

The aging instruction is a control instruction for aging an artificial scale sample. In some embodiments, the aging instruction includes aging parameter such as aging temperature, aging time, aging pressure, or the like. In some embodiments, the processor may determine the aging instruction by querying the second preset table based on the reservoir parameter. The second preset table includes a correspondence between the reservoir parameter and the aging instruction. The second preset table may be determined based on historical data or priori experience.

In some embodiments, when the aging device includes an ultrasonic device, the aging instructions further include ultrasonic parameters. For more information on this section, please refer to the content below.

In some embodiments, the aging temperature is equal to the actual reservoir temperature, and the aging pressure is equal to the actual reservoir pressure, in order to maximize the replication of the actual scale sample. The aging time may be obtained based on priori experience.

In some embodiments, the aging device is further configured to fill an artificial scale sample in a hollow sand-filled pipe based on reservoir parameter, and seal and then aging the artificial scale sample based on the aging parameter to obtain a composite scale sample. The aging parameter includes aging temperature, aging time, aging pressure, or the like.

A hollow sand-filled pipe is a device configured to hold an artificial scale sample. In some embodiments, the artificial scale sample, along with other components to be loaded (e.g., catalyst, reservoir rock, etc.), may be loaded into a hollow sand-filled pipe along a predetermined loading manner (e.g., compaction loading, loose loading, wet loading, etc.) based on reservoir parameters and aging treatment may be performed at the temperature, pressure, and time required for the compaction loading to ultimately form a composite scale sample.

In some embodiments of the present disclosure, the aging device is capable of realistically simulating the high temperature and high pressure environment at the well bottom by placing the artificial scale samples in the hollow sand-filled pipe and sealing and aging them, which helps to enhance the similarity between the composite scale samples and the actual scale sample, and facilitates the subsequent research and anti-scaling technology development.

In some embodiments, the aging time may be 30 d-180 d.

In some embodiments, the aging time may be at least one of 25 d-190 d, 25 d-180 d, 25 d-150 d, 25 d-120 d, 25 d-90 d, 25 d-60 d, 30 d-190 d, 30 d-180 d, 30 d-150 d, 30 d-120 d, 30 d-90 d, 30 d-60 d, 60 d-190 d, 60 d-180 d, 60 d-150 d, 60 d-120 d, 60 d-90 d, 90 d-190 d, 90 d-180 d, 90 d-150 d, 90 d-120 d, 120 d-190 d, 120 d-180 d, 120 d-150 d, 150 d-190 d, 150 d-180 d, 180 d-190 d.

In some embodiments, the aging time may also be at least one of 25 d, 30 d, 60 d, 90 d, 120 d, 150 d, 180 d, 190 d.

Operation 270, aging the artificial scale sample based on the aging instruction to obtain a composite scale sample.

In some embodiments, the aging device may age the artificial scale sample based on an aging temperature and an aging pressure. When reaching the aging time, the aging is completed to obtain a composite scale sample.

In some embodiments of the present disclosure, the process starts with an actual scale sample, followed by qualitative and quantitative analyses of the actual scale sample, then, the formulation of the composite scale sample is set up using the results from these analyses, which ensures that the flocculation type and composition of the composite scale sample closely match those of the actual scale sample. As a result, it is facilitating for the research and development results obtained from the study of the causes of scale samples, the injection well plugging mechanism, and the descaling mechanism, and the design and validation of descaling programs to be more in line with actual conditions, which is more effective. At the same time, because the composite scale sample may be replicated for many times indoors for the corresponding actual scale sample, it is conducive to controlling the consistency of the composite scale samples, which is conducive to promoting reproducibility experiments in the research process of the cause of the scale sample, the plugging mechanism of the injection well, the descaling mechanism, and designing and validation of the descaling program, thereby facilitating the validation of the research results. In addition, the composite scale sample is obtained by replicating indoors, which is low-cost and short time-consuming, and is conducive to the reduction of the cost of research on the cause of scale samples, the plugging mechanism of injection well, the descaling mechanism, and designing and validation of the descaling program.

In some cases, the composition of the flocculent obtained from actual preparation is similar to that of the flocculent in the actual scale sample, but the difference in micro and/or macro structure and scale sample parameter is large (e.g., the difference in scale sample parameter between the flocculent of the actual scale sample and the flocculent of the composite scale sample is greater than a preset difference threshold), resulting in the inability to accurately simulate the actual scale with the artificial scale sample.

To ensure that the artificial scale sample accurately simulate the actual scale sample, in some embodiments, the formulation instruction also includes adjustment parameter.

In some embodiments, the processor is further configured to determine, based on the scale sample parameter of the actual scale sample, an adjustment parameter of the flocculent; the formulation device is further configured to: prepare the flocculent in the composite scale sample based on the flocculation type of the actual scale sample and the polymer composition of the flocculent in the actual scale sample; adjust the scale sample parameter of the flocculent based on the adjustment parameter; and add ingredients other than the flocculent to the flocculent based on the components of the actual scale sample and the content of each component, to obtain the artificial scale sample.

The adjustment parameter is a parameter used to make the flocculent within the artificial scale sample more similar to the flocculent within the actual scale sample. In some embodiments, the adjustment parameter includes pH, temperature, stirring speed, additive type and concentration, reaction time, or the like.

In some embodiments, the formulation device may provide, based on the polymer composition of the flocculent in the actual scale sample, polymer monomer or oligomer, cross-linker, initiator, and other additive, which are mixed to initiate the cross-linking of the polymer monomer or oligomer to gum up to form the consolidant; and then subsequently treatment is performed on the consolidant based on the flocculation type of the actual scale sample. For example, when the flocculation type is a partially oxidized degraded flocculation system, the oxidative degradation is carried out on the consolidant by means of metal ions and/or strong oxidizing substances, and when the degraded and oxidized consolidant has the same or similar scale sample parameter as the actual scale sample, the degraded and oxidized consolidant is taken out and cleaned to obtain the flocculent. For example, if the flocculation type is a poorly dissolved flocculation system, the corresponding solvent is selected to dissolve and treat the consolidant, and when the treated consolidant has the same or similar scale sample parameter as the actual scale sample, the treated consolidant is taken out and cleaned to obtain the flocculent.

According to some embodiments of the present disclosure, the formulation device adjusts the scale sample parameter of the flocculent based on an adjustment parameter. For example, if the scale sample parameter of the flocculent (e.g., viscosity) deviate from the expected value, the formulation device may adjust the aggregation state of the flocculent by varying the stirring speed or the reaction time according to the adjustment parameter determined by the processor.

In some embodiments, the processor may determine an adjustment parameter of the flocculent based on the scale sample parameter of the actual scale sample. For example, the processor may obtain scale sample parameter of the actual scale sample such as the viscosity of the flocculent within the actual scale sample and compare the scale sample parameter with artificially prepared flocculent. When the viscosity of the artificially prepared flocculent is too low, the adjustment parameter may be the appropriate addition of a coagulant; when the viscosity of the artificially prepared flocculent is too high, the adjustment parameter may be controlling the formulation device to mechanically break the flocculent until the viscosity of the flocculent in the artificial scale sample is equal to the viscosity of the actual scale sample.

In some embodiments, when the adjustment parameter is determined, the processor may send the adjustment parameter to the formulation device to adjust the scale sample parameter of the flocculent. After the adjustment is completed, ingredients other than flocculent (e.g., water, crude oil, silica, metal ion salts, etc.) are added into the flocculent and mixed to obtain an artificial scale sample.

It should be noted that the scale sample parameter of the actual scale sample in the present disclosure refers to a parameter of the flocculent separated from the actual scale sample, such as a viscosity of the flocculent separated from the actual scale sample, and not a parameter of the whole actual scale sample.

In some embodiments, the formulation device further includes a first collection device; the processor is further configured to: control the first collection device to collect a preliminary flocculent sample before adjusting the scale sample parameter of the flocculent, and obtain preliminary sampling data based on the analysis device; determine, based on the preliminary sampling data, the flocculation type, a consolidant composition, a reaction medium parameter, and a surface area to volume ratio of a consolidant, a scale sample parameter change sequence; determine, based on the scale sample parameter change sequence and the scale sample parameter, a scale sample sampling time; in response to reaching the scale sample sampling time, control the first collection device to collect a late flocculent sample and obtain late sampling data based on the analysis device; and in response to the late sampling data meeting a completion condition, obtain the flocculent based on the formulation device.

The first collection device is a device that collects a preliminary flocculent sample. In some embodiments, the first collection device includes a robotic arm and a sampling needle, a sampling pump, or the like mounted at the end of the robotic arm.

The preliminary sampling data is sample data from a preliminary flocculent sample. The preliminary flocculent sample means a sample of flocculent extracted from flocculent that has not yet been adjusted based on the adjustment parameter. The processor may control the first collection device to collect the preliminary flocculent sample and obtain the preliminary sampling data based on the analysis device.

The scale sample parameter change sequence is a predicted sequence including predicted scale sample parameters at a plurality of future time points. The scale sample parameter change sequence may reflect changes in scale sample parameter of the preliminary flocculent sample over time during the adjustment process.

The reaction medium parameter are parameters of media properties that affect the flocculation reaction, such as the contents of metal ions, oxidizing substances, and solvents. Parameters such as preliminary sampling data, flocculation type, consolidant composition, reaction medium parameters, and surface area to volume ratio of the consolidant may be obtained by the analysis device.

In some embodiments, the processor may determine a scale sample parameter change sequence via a duration prediction model.

In some embodiments, the duration prediction model may be a machine learning model. For example, any one or combination of recurrent neural network (RNN) model or other customized model structures, etc.

In some embodiments, the inputs to the duration prediction model are preliminary sampling data, flocculation type, consolidant composition, reaction medium parameter, and surface area to volume ratio of the consolidant, and the output of the duration prediction model is a scale sample parameter change sequence. The duration prediction model may be obtained by training a plurality of first training samples with the first labels. The training process includes obtaining a plurality of first training samples with the first labels to form a first training sample set, and executing a plurality of iterations based on the first training sample set. The at least one round of iteration includes: selecting the one or more first training samples from the first training sample set, inputting the one or more first training samples into the initial duration prediction model, obtaining one or more model prediction outputs corresponding to the one or more first training samples; substituting the one or more model prediction outputs corresponding to the one or more first training samples and the first labels corresponding to the one or more first training samples into a formula for a predefined loss function, calculating a value of the loss function; according to the value of the loss function, iteratively updating model parameter of the initial duration prediction model until an end-of-iteration condition is satisfied, ending the iteration, and obtaining the trained duration prediction model. The iteratively updating the model parameters of the initial duration prediction model may be performed in a variety of ways, for example, the updating may be performed based on the gradient descent manner. The end-of-iteration condition may include the loss function converging or the count of iteration reaching the iteration count threshold, etc.

In some embodiments, the first training sample may be obtained based on historical data, and the first label may be automatically labeled by the system based on the historical records.

The scale sample sampling time is the time of sampling from flocculent that have been adjusted based on the adjustment parameter to obtain late sampling data.

The data type and acquisition manner of the late sampling data are similar to that of the preliminary sampling data, with the difference that the preliminary sampling data is the sample data of the flocculent samples before the adjustment, and the late sampling data is the sample data of the flocculent samples after the adjustment.

In some embodiments of the present disclosure, the processor may determine the scale sample sampling time based on a scale sample parameter change sequence and the scale sample parameter. For example, the processor may select, from the scale sample parameter change sequence, a predicted scale sample parameter that is closest to the scale sample parameter of the actual scale sample and determine a future time point corresponding to the predicted scale sample parameter as the scale sample sampling time.

In some embodiments, in response to reaching the scale sample sampling time, the processor may control the first collection device to collect a late flocculent sample and obtain late sampling data based on the analysis device.

In some embodiments, in response to the late sampling data meeting the completion condition, the processor may obtain the adjusted flocculent.

In some embodiments of the present disclosure, a scale sample parameter change sequence is obtained based on data such as preliminary sampling data, and the scale sample parameter change sequence is then used to determine the scale sample sampling time, which can collect the late flocculent sample when the adjustment of preliminary flocculent sample is nearly complete, avoiding the interference and excessive consumption of flocculent caused by blindly collecting large quantities of flocculent samples, thereby enhancing the precision and effectiveness of sample collection.

In some embodiments, the processor is further configured to determine, based on the flocculation type of the actual scale sample, a polymer composition, the components of the actual scale sample, the content of each component of the actual scale sample, and the reservoir parameter, an aging feature changing sequence of the actual scale sample; determine, based on the aging feature changing sequence and an aging feature of the actual scale sample, a target aging time for aging treatment of the artificial scale sample, and send the target aging time to the aging device; and the aging device is further configured to stop aging to obtain the composite scale sample in response to reaching the target aging time.

The aging feature changing sequence is a predicted sequence of the aging features of the actual scale sample changing over time. The aging feature is a feature that changes during aging process, whether or not a change occurs may be determined based on a predicted aging feature changing sequence, i.e., a feature in the predicted aging feature changing sequence with a change amplitude greater than a preset change threshold is the aging feature. The aging feature may include the appearance, mechanical properties, density, degree of polymer degradation, cross-linking density, or the like of the actual scale sample. The aging feature may be obtained by an analysis device.

In some embodiments, the processor may determine an aging feature changing sequence of the actual scale sample based on the flocculation type of the actual scale sample, the polymer composition, the component of the actual scale sample, and the content of each component, and the reservoir parameter.

In some embodiments, the processor may construct a database of vectors based on the simulation data and determine a corresponding aging feature changing sequence by retrieving based on the matching vectors. In some embodiments, the processor may construct target feature vectors based on the flocculation type of the actual scale sample, the polymer composition, the components of the actual scale sample, and the content of each component, and the reservoir parameter. There may be various ways to construct the target feature vector. For example, target feature vectors may be constructed by manners such as Term Frequency-Inverse Document Frequency (TF-IDF), One-Hot, Word2Vec, etc.

The vector database may include a plurality of reference vectors and corresponding reference aging feature changing sequences. Each reference vector may be constructed based on simulation data. The reference vectors are constructed in a similar way to the target feature vectors. The reference aging feature changing sequences may be constructed based on the aging feature changing sequences corresponding to the reference vectors. The simulation data may be obtained by simulation experiments, for example, obtaining a plurality of experimental scale samples, aging the experimental scale samples, and obtaining the aging feature changing sequences as reference aging feature changing sequences during the aging process.

In some embodiments, the processor may determine a target aging feature changing sequence based on similarities between the target feature vector and a plurality of reference vectors in the vector database. For example, the reference vectors whose similarities with the target feature vector satisfy the preset similarity condition are taken as the target vectors, and the reference aging feature changing sequences corresponding to the target vectors are taken as the final target aging feature changing sequence. The preset similarity condition may be set according to the situation. For example, the similarity is maximum, or the similarity is greater than a threshold, etc. For another example, the vector distance is minimum.

The target aging time is a target duration for aging treatment of artificial scale sample.

In some embodiments, the processor may determine a target aging time for aging treatment of the artificial scale sample based on the aging feature changing sequence and the aging feature of the actual scale sample and send the target aging time to the aging device. For example, the processor may compare the predicted aging features included in the aging feature changing sequence with the aging feature of the actual scale sample and select the predicted aging time of the predicted aging feature with the highest similarity as the target aging time.

In some embodiments of the present disclosure, the processor may determine a target aging time based on an aging feature changing sequence and aging feature of the actual scale sample, and automatically stop aging after the target aging time is reached, which helps to realize automation of the system and improves the operation convenience and safety.

In some embodiments, the processor is further configured to obtain a candidate aging parameter; determine, based on an initial scale sample feature, a scale sample related parameter, a scale sample amount, the candidate aging parameter, and the aging feature, a predicted scale sample feature sequence corresponding to the candidate aging parameter by a feature change prediction model; determine an aging parameter and the target aging time based on the predicted scale sample feature sequence and the aging feature; and generate, based on the aging parameter and the target aging time, the aging instruction and send the aging instruction to the aging device.

The scale sample related parameter includes the flocculation type, polymer composition, components of the scale sample, and the content of each component. The scale sample amount is a mass of the artificial scale sample.

The candidate aging parameter is combinations of aging parameter to be selected. In some embodiments, the processor may select historical aging parameter as the candidate aging parameter.

The initial scale sample feature is a scale sample feature of the current artificial scale sample. The scale sample feature may include aging feature and other feature, and the other feature is those that remain unchanged or substantially unchanged during aging process. The initial scale sample feature may be obtained by an analysis device.

The predicted scale sample feature sequence is a predicted sequence including a plurality of predicted scale sample features at a plurality of future time points.

In some embodiments, the processor may determine a predicted scale sample feature sequence corresponding to the candidate aging parameter based on the initial scale sample feature, scale sample related parameter, scale sample amount, candidate aging parameter, and aging feature by a feature change prediction model.

The feature change prediction model is a model configured to determine the predicted scale sample feature sequence corresponding to candidate aging parameter. In some embodiments, the feature change prediction model may be a machine learning model, such as a recurrent neural network (RNN) model, or the like.

In some embodiments, inputs to the feature change prediction model include an initial scale sample feature, a scale sample related parameter, a scale sample amount, a candidate aging parameter, and an aging feature. The output of the feature change prediction model is a predicted scale sample feature sequence.

In some embodiments, the feature change prediction model is obtained by training, the training comprising: obtaining a plurality of second training samples with second labels to constitute a training sample set and performing at least one round of iterations based on the training sample set. The second training samples comprise a sample initial scale sample feature of a sample scale sample, a sample scale sample related parameter, a sample scale sample amount, a sample aging parameter, and a sample aging feature, and the second labels comprise a scale sample feature sequence corresponding to the sample scale sample.

The sample scale sample refers to a scale sample used to provide data source for a training sample set. The scale sample feature sequence refers to a sequence formed by the scale sample features of the sample scale sample at a plurality of time points.

In some embodiments, the training sample set may be obtained through simulation experiments. For example, a plurality of sample scale sample is obtained for performing aging experiments, and the scale sample features are monitored at different time points during the aging process to construct a scale sample feature sequence.

The processor may acquire a plurality of sample scale samples and obtain the initial scale sample features, scale sample related parameters, sample scale sample amount, and sample aging features of the sample scale samples via the analysis device, and the sample aging parameter may be preset aging parameter. The aging device may age the sample scale sample based on the sample aging parameter, monitor the scale sample features at different time points during the aging process via the analysis device, construct a scale sample feature sequence, and determine the scale sample feature sequence as the second label for the second training sample. By repeating the above operation, a plurality of second training samples with the second labels may be obtained. For more information on obtaining a plurality of experimental scale samples, please refer to the following related description.

In some embodiments, at least one round of iteration includes: selecting at least one second training sample from the training sample set and inputting the at least one second training sample to an initial feature change prediction model to obtain an output of the initial feature change prediction model corresponding to the at least second one training sample; determining, based on the output of the initial feature change prediction model corresponding to the at least one second training sample and the second training label of the at least one training sample, a loss function; iteratively updating model parameters of the initial feature change prediction model based on the loss function; and in response to satisfying an end-of-iteration condition, ending iteration to obtain the feature change prediction model.

In some embodiments, the processor may input a plurality of second training samples with second training labels into the initial feature change prediction model, construct a loss function using the second labels and the model prediction output, and iteratively update the initial feature change prediction model based on the loss function. The model training is completed when the preset conditions are met to obtain the trained feature change prediction model. The preset conditions may include a convergence of the loss function, the number of iterations reaching a threshold, etc.

In some embodiments, the processor may determine the aging parameter and the target aging time based on a predicted scale sample feature sequence and an aging feature. The target aging time is similar to the aging time, and the difference is that the target aging time is less than the aging time, which may reduce the time consumed by aging.

For example, the processor may filter the predicted scale sample feature sequences to retain only the predicted scale sample feature sequences that satisfy the qualifying conditions. From the qualified predicted scale sample feature sequences, a set of predicted scale sample feature sequences with the fastest aging of the artificial scale sample is selected. The time at which the aging of the set of predicted scale sample feature sequences is completed is determined as the target aging time, and a candidate aging parameter for the set of predicted scale sample feature sequences is determined as the aging parameter. The qualifying condition may be defined as follows: within the predicted scale sample feature sequence, there are scale sample features whose deviation from the scale sample feature of the actual scale sample is less than a predetermined threshold (e.g., ±10%).

In some embodiments of the present disclosure, by introducing a feature change prediction model, it is possible to predict a scale sample feature change sequence corresponding to a candidate aging parameter based on an initial scale sample feature, a scale sample related parameter, a scale sample amount, a candidate aging parameter, and an aging feature, which optimizes the process of determining aging parameter and target aging time. An aging instruction is generated and sent to the aging device, realizing the dynamic control of the aging process.

In some embodiments, a manner of obtaining a sample scale sample includes controlling, based on a scale sample collection weight, a number of sampling sites, and a first sampling period, the first collection device to obtain the sample scale sample.

The number of sampling sites refers to the number of different sampling locations selected for scale sample collection at the well bottom or within an experimental scale sample.

The first sampling period refers to the time interval for scale sample collection within a certain time range.

In some embodiments, the preparation system for a well bottom composite scale sample may control the first collection device to obtain a sample scale sample based on scale sample collection weight, a number of sampling sites, and a first sampling period.

In some embodiments, the scale sample collection weight, the number of sampling sites, and the first sampling period may be preset based on prior experience.

In some embodiments, the scale sample collection weight and the number of sampling sites are positively correlated to the weight and volume of the scale sample, and the first sampling period is negatively correlated to the aging intensity. The aging intensity is determined based on the sample aging parameter.

The aging intensity is used to characterize the degree of aging of the scale sample by the aging parameter. The more intense the aging parameter (e.g., higher temperature and pressure), the faster the aging rate and the greater the aging intensity for the scale sample.

In some embodiments, the aging intensity is determined based on the sample aging parameter. For example, when the temperature and pressure in the sample aging parameter are low, the aging intensity of the sample is small and the aging process of the scale sample is slower; at this time, a larger first sampling period may be employed to save manpower and material resources; when the temperature and pressure in the sample aging parameter are higher, the aging process of the scale sample is faster. In order to ensure that the scale sample is not over-aging, a smaller first sampling period may be employed to monitor the state of the sample more frequently.

In some embodiments of the present disclosure, the scale sample collection weight and the number of sampling sites are positively correlated with the weight and volume of the scale sample, enabling flexible adjustment of the scale sample collection weight and the number of sampling sites based on the weight and volume of the scale sample, thereby improving the representativeness of the scale sample and the accuracy of the data; the first sampling period is negatively correlated with the aging intensity, so reasonable adjustments to the first sampling period can be made to avoid overly frequent or sparse sampling, thus enhancing sampling efficiency and data timeliness.

According to some embodiments of the present disclosure, the scale sample collection weight, the number of sampling sites, and the sampling period are controlled to ensure that the samples obtained are representative, thereby enhancing the predictability of changes in the aging features of the samples.

In some embodiments, the aging device may include an ultrasonic device. The ultrasonic device may accelerate the aging process of the scale sample through the high-frequency vibration and cavitation effects of ultrasound.

In some embodiments, when the aging device includes an ultrasonic device, candidate aging parameter and aging parameter may include an ultrasonic parameter. For example, the ultrasonic parameters may include ultrasonic frequency, power, or the like.

According to some embodiments of the present disclosure, by introducing an ultrasonic device and a corresponding ultrasonic parameter during the aging process, it is possible to accelerate the aging process of the scale samples and to improve the aging uniformity.

In some embodiments, the aging device further includes a second collection device, and the processor is further configured to: determine a second sampling period and a sampling parameter during an aging process and sending the second sampling period and the sampling parameter to the second collection device; control the second collection device to sample artificial scale sample during the aging process in accordance with the sampling parameter based on the second sampling period, to obtain an aging scale sample, and transmit the aging scale sample to the analysis device; control the analysis device to analyze the aging scale sample to determine aging scale sample data; generate, based on the aging scale sample data, the predicted scale sample feature sequence, the scale sample related parameter, and a current aging parameter, an adjustment instruction, and send the adjustment instruction to the aging device to adjust the aging parameter of the aging device.

The structure and function of the second collection device is similar to that of the first collection device, with the difference being that the second collection device is sampling an artificial scale sample from the aging process, and he first collection device is sampling a flocculent sample.

In some embodiments, the processor may determine the second sampling period and the sampling parameter during the aging process and send them to the second collection device. The second sampling period is acquired in a similar manner as the first sampling period, and the duration of the second sampling period may be the same or different from the duration of the first sampling period. The sampling parameters may include sampling positions and a sampling amount corresponding to each sampling position.

In some embodiments, the processor may control, based on the second sampling period, the second collection device to sample the artificial scale sample during aging process in accordance with the sampling parameters to obtain the aging scale sample.

In some embodiments, the processor may analyze the aging scale sample by an analysis device to determine the aging scale sample data; and the aging scale sample data may include density, viscosity, degree of polymer degradation, cross-linking density, mechanical properties, electrical conductivity, chemical composition, or the like.

In some embodiments, in response to satisfying an adjustment condition, the processor may generate an adjustment instruction. The adjustment condition may be that the difference between the sampled scale sample feature acquired by the analysis at that time point and the predicted scale sample feature acquired by the feature change prediction model at that time point is greater than a preset threshold, etc. The preset threshold may be determined by manual experience.

In some embodiments, the processor may generate an adjustment instruction based on the aging scale sample data, the predicted scale sample feature sequence, the scale sample related parameters, and the current aging parameter.

The adjustment instruction is an instruction to adjust the aging parameter of the aging device. The adjustment instruction includes the parameter to be adjusted in the aging parameter, the direction of adjustment, and the magnitude of adjustment. For example, the adjustment instruction may specify a temperature increase of 5° C. in the next 2 min.

In some embodiments, the processor may construct a database of vectors based on the simulation experiments and determine corresponding adjustment instruction based on vector matching. In some embodiments, the processor may construct a target feature vector based on the aging scale sample data, the predicted scale sample feature sequence, the scale sample related parameter, and the current aging parameter. The construction of the target feature vector may utilize various manners. For example, the target feature vector may be constructed using manners such as TF-IDF, One-Hot, Word2Vec, or the like. The predicted scale sample feature sequence is the output of the feature change prediction model. For more on the scale sample related parameters and current aging parameter, please see related description above.

The vector database may include a plurality of reference vectors and corresponding reference adjustment instruction. Each of the reference vectors may be constructed based on simulation experiments. The reference vectors are constructed in the same way as the target feature vectors. For example, the reference adjustment instruction may be determined based on simulation. For example, obtaining a plurality of experimental scale samples for aging, adjusting them individually using a plurality of sets of preset adjustment instructions, and collecting aging scale sample at the next time point after the adjustments for analysis, obtaining the scale sample feature for the next time point, and comparing the scale sample feature with the predicted scale sample feature for the same time point in the predicted scale sample feature sequence outputted by the feature change prediction model, in response to determining that the difference is less than a preset difference threshold, determining the corresponding parameter adjustment data to be reference parameter adjustment data corresponding to the reference vector.

In some embodiments, the processor may determine a target adjustment instruction based on the similarity between the target feature vector and a plurality of reference vectors in the vector database. For example, reference vectors whose similarities to the target feature vector satisfy the preset similarity condition may be selected as target vectors, and the corresponding reference adjustment instructions are taken as the final target adjustment instructions. The similarity preset condition may be set as appropriate. For example, the similarity being maximum, or similarity greater than a threshold, etc. For example, the vector distance being minimum.

According to some embodiments of the present disclosure, adjusting the aging parameter of the aging device by adjustment instructions allows real-time regulation of the aging process based on the actual situation, which helps to improve the similarity between the composite scale sample and the actual scale sample, and to enhance the aging performance.

The present disclosure also provides composite scale samples of organic matter system obtained by using a preparation method for a well bottom composite scale sample.

In order to better illustrate the technical solutions of the embodiments of the present disclosure, the composite scale samples corresponding to the scale samples of the JZ9-3 oil well of a certain oil field are prepared as an example, and the specific operations of preparation are as follows.

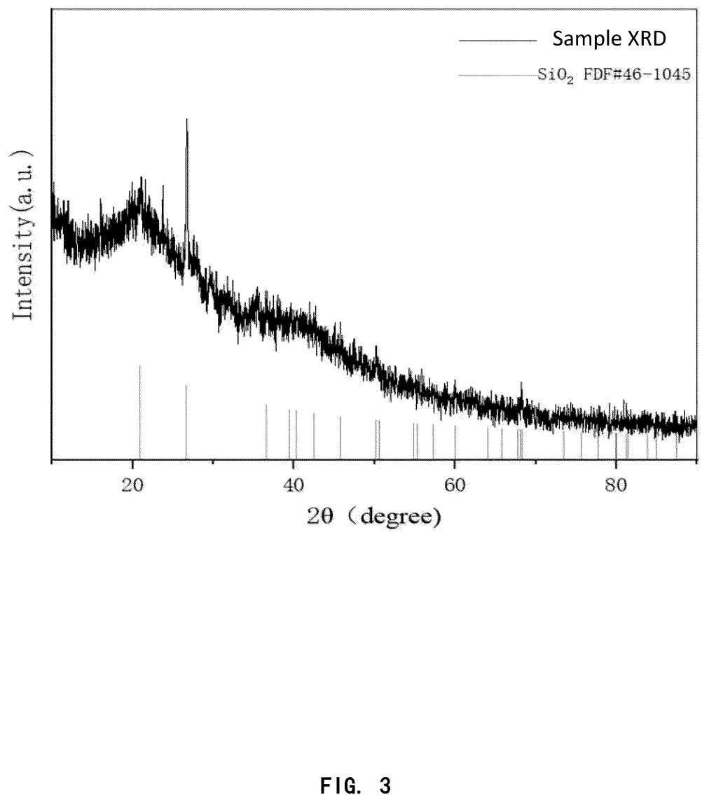

Scale samples are taken for analyzing the scale sample components of the target JZ9-3 oil well, and the taken scale samples are divided into a plurality of parts for morphological analysis, compositional qualitative analysis, and compositional quantitative analysis. The morphological analysis is mainly carried out using scanning electron microscope, the compositional qualitative analysis is carried out using XRD and infrared spectroscopy, and the compositional quantitative analysis is carried out using dry distillation at 110° C., extraction of crude oil with hexane, high-temperature thermal degradation of organic matter at 650° C., and multi-point scanning electron microscope energy spectrum analysis. The test results are shown in FIGS. 3-15, and the specific content of each component in the obtained scale sample is shown in Table 1.

TABLE 1

| | Scale | Scale | Scale | Scale | |
|---|---|---|---|---|---|
| Test item | sample 1 | sample 2 | sample 3 | sample 4 | average |
| moisture | 89.03% | 89.03% | 89.08% | 89.09% | 89.05% |
| content | | | | | |
| oil content | 0.995% | 0.980% | 0.979% | 0.925% | 0.995% |
| organic | 8.81% | 8.65% | 8.77% | 8.87% | 8.81% |
| matter | | | | | |
| content | | | | | |
| SiO₂ | 8.339% | 9.648% | 8.507% | 8.087% | 8.339% |
| Fe ion | 0.003% | 0.003% | 0.004% | 0.003% | 0.003% |
| Mg ion | 0.015% | 0.015% | 0.015% | 0.015% | 0.015% |
| Ca ion | 0.231% | 0.278% | 0.242% | 0.225% | 0.231% |
| Cr ion | / | / | / | / | / |

Quartz sand simulating SiO₂, soluble iron salts providing iron ions, soluble magnesium salts providing magnesium ions, soluble calcium salts providing calcium ions, and soluble chromium salts providing chromium ions, the formation water of the target oil field for sampling, and the degassed and dehydrated crude oil from the target reservoir for sampling are prepared indoors.

The organic matter in the scale samples was qualitatively analyzed by infrared spectroscopy, indicating the presence of characteristic absorption peaks of groups such as amide group (—CONH₂), carboxyl group (—COO—), and —CH₂— group, exhibiting obvious characteristics of partially hydrolyzed polyacrylamide (HPAM). It may be determined that the colloidal material in the scale sample is mainly cross-linked polyacrylamide. A part of the organic matter in the scale samples is sampled for the apparent viscosity test, and a viscosity retention rate is only 25% of the original system, and at the same time, the scale samples have a high content of Fe ions, and the analysis suggests that the organic matter has been subjected to oxidized degraded flocculation system.

The polymer solution with a target concentration is first prepared indoors, after completely dissolving, and then left to stand for 24 h. The polymer solution is oxidized using Fe³⁺ as required to has the viscosity feature of the scale sample organic matter, i.e. 25% of the target system.

Based on the determined components of the scale sample, after measuring one by one according to the components in Table 1, the organic matter and other substances other than the organic matter are added according to the proportion, and placed in a stirring and mixing tank for stirring and mixing; furthermore, a hollow sand-filled pipe is configured to fill the mixed scale samples in above operation, and the pressure of the hollow sand-filled pipe reaches the bottom pressure of the target oil well, and is kept and closed for use.

The high-pressure scale sample sand-filled pipe is statically preserved in a constant temperature box at reservoir temperature, the scale samples are prepared by aging; after aging for the target time, the pressure was relieved in the sand-filled pipe, and the scale samples were removed.

One or more embodiments of the present disclosure provide a preparation method for a well bottom composite scale sample, including the following steps: obtaining an actual scale sample of a polymer injection well and a reservoir parameter of the reservoir in which the polymer injection well is located; performing a qualitative analysis on the actual scale sample to determine the flocculation type of the actual scale sample and a scale sample parameter; performing a quantitative analysis on the actual scale sample to determine components of the actual scale sample and a content of each component; formulating an artificial scale sample according to the flocculation type of the actual scale sample, the components of the actual scale sample, and the content of each component; and aging the artificial scale sample according to the reservoir parameter of the polymer injection well to obtain a composite scale sample.

In some embodiments, the flocculation type includes: a partially oxidized degraded flocculation system, a consolidated conformance control system, and a poorly dissolved flocculation system; and the scale sample parameter includes the viscosity of the actual scale sample.

In some embodiments, the qualitative analysis includes a compositional qualitative analysis and a morphological qualitative analysis of the actual scale sample, and the flocculation type is determined based on the compositional qualitative analysis and the morphological qualitative analysis of the actual scale sample.

In some embodiments, the compositional qualitative analysis further includes analyzing the polymer composition of the flocculent in the actual scale sample.

In some embodiments, the formulating an artificial scale sample according to a flocculation type of the actual scale sample, components of the actual scale sample, and a content of each component includes the following operations: preparing a flocculent in the composite scale sample according to a flocculation type of the actual scale sample and a polymer composition of a flocculent in the actual scale sample; setting an adjustment parameter of the composite scale sample according to the scale sample parameter of the actual scale sample; adjusting the scale sample parameter of the flocculent to the adjustment parameter according to the adjustment parameter; and adding ingredients other than the flocculent to the flocculent according to the components of the actual scale sample and the content of each component to obtain the artificial scale sample.

One or more embodiments of the present disclosure provide a preparation method for a well bottom composite scale sample, wherein an artificial scale sample is filled in a hollow sand-filled pipe according to reservoir parameter, and the artificial scale sample is aged after sealing and setting up aging condition.

In some embodiments, the reservoir parameter includes an actual reservoir temperature and an actual reservoir pressure.

In some embodiments, the aging time is 30 d-180 d.

It should be noted that the above descriptions are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or collocation of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer-readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A preparation system for a well bottom composite scale sample, comprising an analysis device, a processor, a formulation device, and an aging device, wherein the processor is configured to:
obtain an actual scale sample of a polymer injection well and a reservoir parameter of a reservoir in which the polymer injection well is located;
perform a qualitative analysis on the actual scale sample by the analysis device to determine a flocculation type and a scale sample parameter of the actual scale sample;
perform a quantitative analysis on the actual scale sample by the analysis device to determine components of the actual scale sample and a content of each component of the actual scale sample;
generate a formulation instruction based on the flocculation type of the actual scale sample, the components of the actual scale sample, and the content of each component of the actual scale sample, and send the formulation instruction to the formulation device to cause the formulation device to formulate an artificial scale sample; and
generate, based on the reservoir parameter, an aging instruction, and send the aging instruction to the aging device to cause the aging device to age the artificial scale sample to obtain a composite scale sample.

2. The system of claim 1, wherein the flocculation type comprises a partially oxidized degraded flocculation system, a consolidated conformance control system, and a poorly dissolved flocculation system; and the scale sample parameter comprises a viscosity of the actual scale sample.

3. The system of claim 1, wherein the qualitative analysis comprises a compositional qualitative analysis and a morphological qualitative analysis, and the analysis device is further configured to:
determine, by the compositional qualitative analysis and the morphological qualitative analysis, the flocculation type of the actual scale sample.

4. The system of claim 3, wherein the analysis device is further configured to:
determine a polymer composition of a flocculent in the actual scale sample by the compositional qualitative analysis; and
the processor is further configured to:
generate the formulation instruction based on the flocculation type of the actual scale sample, the polymer composition of the flocculent in the actual scale sample, the components of the actual scale sample, and the content of each component of the actual scale sample, and send the formulation instruction to the formulation device.

5. The system of claim 4, wherein the formulation instruction further comprises an adjustment parameter,
the processor is further configured to:
determine, based on the scale sample parameter of the actual scale sample, an adjustment parameter of the composite scale sample; and
the formulation device is further configured to:
prepare, based on the flocculation type of the actual scale sample and the polymer composition of the flocculent in the actual scale sample, a flocculent in the composite scale sample;
adjust, based on the adjustment parameter, the scale sample parameter of the flocculent; and
add ingredients other than the flocculent into the flocculent to obtain the artificial scale sample based on the components of the actual scale sample and the content of each component of the actual scale sample.

6. The system of claim 5, wherein the formulation device further comprises a first collection device, and the processor is further configured to:
control the first collection device to collect a preliminary flocculent sample before adjusting the scale sample parameter of the flocculent, and obtain preliminary sampling data based on the analysis device;
determine, based on the preliminary sampling data, the flocculation type, a consolidant composition, a reaction medium parameter, and a surface area to volume ratio of a consolidant, a scale sample parameter change sequence;
determine, based on the scale sample parameter change sequence and the scale sample parameter, a scale sample sampling time;
in response to reaching the scale sample sampling time, control the first collection device to collect a late flocculent sample and obtain late sampling data based on the analysis device; and
in response to the late sampling data meeting a completion condition, obtain the flocculent based on the formulation device.

7. The system of claim 1, wherein the aging device is further configured to:
fill the artificial scale sample into a hollow sand-filled pipe according to the reservoir parameter and then seal the hollow sand-filled pipe, and age the artificial scale sample in the hollow sand-filled pipe based on an aging parameter to obtain the composite scale sample.

8. The system of claim 1, wherein the processor is further configured to:
determine, based on the flocculation type of the actual scale sample, a polymer composition, the components of the actual scale sample, the content of each component of the actual scale sample, and the reservoir parameter, an aging feature changing sequence of the actual scale sample;
determine, based on the aging feature changing sequence and an aging feature of the actual scale sample, a target aging time for aging treatment of the actual scale sample, and send the target aging time to the aging device; and
the aging device is further configured to:
in response to reaching the target aging time, stop aging to obtain the composite scale sample.

9. The system of claim 8, wherein the processor is further configured to:
obtain a candidate aging parameter;
determine, based on an initial scale sample feature, a scale sample related parameter, a scale sample amount, the candidate aging parameter, and the aging feature, a predicted scale sample feature sequence corresponding to the candidate aging parameter by a feature change prediction model, the feature change prediction model being a machine learning model;
determine an aging parameter and the target aging time based on the predicted scale sample feature sequence and the aging feature;
generate, based on the aging parameter and the target aging time, the aging instruction and send the aging instruction to the aging device.

10. The system of claim 9, wherein the feature change prediction model is obtained by training, the training comprising:
obtaining a plurality of training samples with labels to constitute a training sample set, and performing at least one round of iterations based on the training sample set, the training samples comprising a sample initial scale sample feature of a sample scale sample, a sample scale sample related parameter, a sample scale sample amount, a sample aging parameter, and a sample aging feature, and the labels comprising a sample predicted scale feature sequence corresponding to the sample scale sample;
wherein the at least one round of iteration comprises:
selecting at least one training sample from the training sample set and inputting the at least one training sample to an initial feature change prediction model to obtain an output of the initial feature change prediction model corresponding to the at least one training sample;
determining, based on the output of the initial feature change prediction model corresponding to the at least one training sample, and the training label of the at least one training sample, a loss function;
iteratively updating model parameters of the initial feature change prediction model based on the loss function; and
in response to satisfying an end-of-iteration condition, ending iteration to obtain the feature change prediction model.

11. The system of claim 10, wherein the formulation device further comprises a first collection device, a manner of obtaining the sample scale sample comprises:
controlling, based on a scale sample collection weight, a number of sampling sites, and a first sampling period, the first collection device to obtain the sample scale sample.

12. The system of claim 11, wherein the scale sample collection weight and the number of sampling sites are positively correlated to a weight and a volume of the sample scale sample, and the first sampling period is negatively correlated to an aging intensity, the aging intensity being determined based on the sample aging parameter.

13. The system of claim 9, wherein the aging device further comprises an ultrasonic device, and the candidate aging parameter and the aging parameter comprise an ultrasonic parameter.

14. The system of claim 9, wherein the aging device further comprises a second collection device,
and the processor is further configured to:
determine a second sampling period and a sampling parameter during an aging process and sending the second sampling period and the sampling parameter to the second collection device;
control the second collection device to sample artificial scale sample during the aging process in accordance with the sampling parameter based on the second sampling period, to obtain an aging scale sample, and transmit the aging scale sample to the analysis device;
control the analysis device to analyze the aging scale sample to determine aging scale sample data;
generate, based on the aging scale sample data, the predicted scale sample feature sequence, the scale sample related parameter, and a current aging parameter, an adjustment instruction, and send the adjustment instruction to the aging device to adjust the aging parameter of the aging device.

15. The system of claim 7, wherein the reservoir parameter comprises an actual reservoir temperature and an actual reservoir pressure.

16. The system of claim 1, wherein an aging time for performing aging is within a range of 30 d to 180 d.

17. A composite scale sample of an organic matter system, wherein the composite scale sample of the organic matter system is prepared according to the preparation system for a well bottom composite scale sample of claim 1.
